(19) United States
(12) Patent Application Publication
(10) Pub. No.: US 2011/0230920 A1
(43) Pub. Date: Sep. 22, 2011

(54) SPINAL FIXATION APPARATUS AND METHODS

(75) Inventors: Josef GOREK, Ross, CA (US); Scott JONES, McMurray, PA (US)

(73) Assignee: K2M, INC., Leesburg, VA (US)

(21) Appl. No.: 13/052,669

(22) Filed: Mar. 21, 2011

Related U.S. Application Data
(60) Provisional application No. 61/315,447, filed on Mar. 19, 2010.

(51) Int. Cl. A61B 17/86

(52) U.S. Cl. 606/305

(57) ABSTRACT

A bone engaging assembly includes first second bone engaging members. The first bone engaging member defines a first axis and has a first head and a first shaft. The first head defines one or more apertures therethrough. In embodiments, the first head defines a driver interface. The driver interface is defined within the first head and is configured to engage a driving instrument. In one embodiment, the first head includes a post extending from the first head that is configured to engage a rod coupling member. The second bone engaging member defines a second axis. The second bone engaging member defines a second head and a second shaft. The first axis of the first bone engaging member and the second axis of the second bone engaging member define an angle therebetween when the second bone engaging member is positioned within the one or more apertures of the first head.

### CROSS REFERENCE TO RELATED APPLICATIONS

This application claims priority to, and the benefit of, U.S. Provisional Patent Application Ser. No. 61/315,447 filed Mar. 19, 2010, the entire contents of which are incorporated by reference herein.

### BACKGROUND

1. Technical Field

The present disclosure relates generally to orthopedic surgery with particular regard to spinal surgery. Specifically, the present disclosure relates to apparatuses and methods for apparatus and methods for supplemental spinal screw fixation.

2. Description of Related Art

The spinal column is a complex system of bones and connective tissues that provide support for the human body and protection for the spinal cord and nerves. The adult spine is comprised of an upper and lower portion. The upper portion contains 24 discrete bones, which are subdivided into three areas including 7 cervical vertebrae, 12 thoracic vertebrae and 5 lumbar vertebrae. The lower portion is comprised of the sacral and coccygeal bones. The cylindrical shaped bones, called vertebral bodies, progressively increase in size from the upper portion downwards to the lower portion.

An intervertebral disc along with two posterior facet joints cushion and dampen the various translational and rotational forces exerted upon the spinal column. The intervertebral disc is a spacer located between two vertebral bodies. The facets provide stability to the posterior portion of adjacent vertebrae. The spinal cord is housed in the canal of the vertebral bodies. It is protected posteriorly by the lamina. The lamina is a curved surface with three main protrusions. Two transverse processes extend laterally from the lamina, while the spinous process extends caudally and posteriorly. The vertebral bodies and lamina are connected by a bone bridge called the pedicle. The spine is a flexible structure capable of a large range of motion. There are various disorders, diseases and types of injury which restrict the range of motion of the spine or interfere with important elements of the nervous system. The problems include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function.

There are many procedures that can be utilized to address the aforementioned conditions. A conventional ventrolateral transpsoas approach has been developed for instrumentation of L1 through L4. With the patient positioned in a 90 degree lateral decubitus position, the spine is accessed laterally through the psoas muscle. Through this approach, morbidity from dural exposure, excessive nerve root retraction, epidural bleeding, and excessive scaring may occur. Additionally, injury to the femoral or genitorfemoral nerves may occur. A second approach is the conventional dorsal approach to the lumbar spine which places the patient in a prone position and, through midline incision over the level of interest, dissects and laterally retracts the dorsal paraspinal muscles. Apart from a significant risk of blood loss, the dural sac and the posterior rami, which lie between the transverse processes lateral to the pars interarticularis and the facet joint capsules, may be injured. Peri-operative pain and post-operative scarring are typically encountered following the dorsal approach. A standard anterior approach is also common for addressing a discectomy procedure and is typically followed up by a posterior instrumentation procedure to secure the anatomy. In this procedure, pedicle screws are typically used for posterior instrumentation, but there can be significant trauma and clinically significant hardware impingement on soft tissues since the locations of the screw heads and the rods are inter/intramuscular. Also, pedicle screws can injure the cephalad facet joint. Less invasive and less bulky fixation approaches are the various versions of transfacet fixation. Since these are intraosseous and transfacet, there are minimal risks for muscle irritation and injury to the cephalad facet joint. The problem with typical facet fixation is the inherent inability of the construct to withstand the flexion experienced by the lumbar segments.

Spinal fixation apparatuses are widely employed in surgical processes for correcting spinal injuries and diseases. When the disc has degenerated to the point of requiring removal, there are a variety of interbody implants that are utilized to take the place of the disc. These include, PEEK interbody spacers, metal cages and cadaver and human bone implants. In order to facilitate stabilizing the spine and keeping the interbody in position, other implants are commonly employed, including longitudinally linked rods secured to coupling elements, which in turn are secured to the bone by spinal bone fixation fasteners such as pedicle screws, hooks, and others. The opposing pair of longitudinally linked rods is commonly disposed along the long axis of the spine via a posterior approach. In lieu of using pedicle screws and rods, a transfacet intrapedicular screw can be utilized. This screw can be manufactured from any biocompatible material, including cobalt chrome, stainless steel, titanium and PEEK (polyetheretherketone).

To meet the problem of withstanding the flexion of the lumbar segments previous attempts have been made with: A) translaminar transfacet fixation whereby the screw stays in the lamina and has good proximal purchase, but weak distal purchase due to only a superficial purchase in the superior facet (the insertion of this device is technically demanding with the hazard of breaching into the spinal canal); B) transfacet fixation, which is a more lateral trajectory versus a cranial-caudad trajectory and has limited proximal and distal fixation; and C) a facet bolt structure which has limited bone purchase both proximally and distally.

Therefore, a need exists for a spinal screw or anchor that provides secure attachment means to the anatomy while also providing posterior support to the surgical construct.

### SUMMARY

The present disclosure is directed to bone engaging assemblies including a first bone engaging member and a second bone engaging member. The first bone engaging member defines a first axis and has a first head and a first shaft. The first head defines one or more apertures therethrough and may be coupled to a rod coupling member. A rod may be secured to the rod coupling member. In embodiments, the first head defines a driving interface. The driving interface is defined within the first head and is configured to engage a driving instrument. In one embodiment, the first head includes a post extending from the first head that is configured to engage a rod coupling member. The second bone engaging member defines a second axis. The second bone engaging member defines a second head and a second shaft. The first axis of the first bone engaging member and the second axis of the second bone engaging member define an angle therebetween when the second bone engaging member is positioned within the one or more apertures of the first head. At least a portion of one of the first and second bone engaging member may be made of commercially pure titanium and at least a portion of the other of the first and second bone engaging member may be made of titanium alloy.

The second head may be in contact with the first head when the second bone engaging member is positioned within the one or more apertures of the first head. The first bone engaging member may be oriented at an acute angle relative to the second bone engaging member. The first bone engaging member defines a first length and the second bone engaging member defines a second length. The first and second lengths may be different. The one or more apertures may be positioned at an angle relative to the first axis of the first bone engaging member and in parallel with the second axis of the second bone engaging member. The first head defines a first surface and one or more second surfaces. The one or more second surfaces may project substantially parabolically from the first surface. The one or more second surfaces may define an internal section and an external section. Boundaries of the internal section define at least a portion of the one or more apertures.

In one embodiment, the first head includes a collar that is integrally formed with the first head. In this embodiment, the one or more apertures are defined within the collar. A plurality of apertures may be defined within the collar and positioned radially about the collar. The collar is longitudinally spaced from the driver interface along the first axis.

In one embodiment, the first head includes a collar that is selectively attachable to the first head. The selectively attachable collar defines the one or more apertures therethrough and a passage therethrough. The passage facilitates the securement of the collar to the first head whereby the screw post extends proximally of the passage. In this embodiment, the one or more apertures are positioned at an angle relative to the passage.

According to one aspect, a method of mounting a bone engaging assembly includes providing a first bone engaging member and a second bone engaging member, the first bone engaging member including a first head and a first shank, the second bone engaging member including a second head and a second shank. The method includes anchoring the first bone engaging member through the facet joint (i.e., through the inferior facet of a cranial vertebra and the superior facet of a caudal vertebra) and into the pedicle of the caudal vertebra, mounting the second head of the second bone engaging member to the first head of the first bone engaging member, and anchoring the second bone engaging member to an adjacent boney structure at the cranial level, such as the pars interarticularis or the pars interarticularis and pedicle of the cranial vertebra. The method may also involve advancing the first bone engaging member into the vertebral body of the caudal vertebra. Alternatively, the first bone engaging member could be directed into the pars interarticularis of a cranial vertebra, with the second bone engaging member mounted through an aperture of the first bone engaging member, through the inferior facet of the cranial vertebra, into the facet of a caudal vertebra or the facet and pedicle or facet and pedicle and vertebral body of the caudal vertebra. The method may include providing a first bone engaging member with a first head including one or more apertures and adjusting the first head of the first bone engaging member so that the position of the one or more apertures are oriented to facilitate a desired trajectory of the second bone engaging member. The method may include cold welding the second bone engaging member to the first bone engaging member upon mounting the second bone engaging member to the first bone engaging member. One step involves positioning the second shank superiorly of the first shank.

In accordance with one aspect, the first bone screw may be placed into the sacrum with the second bone engaging member may be advanced laterally into the sacral ala.

### BRIEF DESCRIPTION OF THE DRAWINGS

The above and other aspects and features of the present disclosure will become more apparent in light of the following detailed description when taken in conjunction with the accompanying drawings in which:

FIG. 1A is a perspective view of one embodiment of a bone engaging assembly in accordance with the present disclosure;

FIG. 1B is an end view of the bone engaging assembly of FIG. 1A;

FIG. 1C is a side cross-sectional view of the bone engaging assembly of FIG. 1B taken along section line 1C-1C;

FIG. 2 is a perspective view of a first bone engaging member of the bone engaging assembly of FIG. 1A;

FIG. 2A is a perspective view of an alternate embodiment of the first bone engaging member of FIG. 2;

FIG. 3 is a perspective view of a second bone engaging member of the bone engaging assembly of FIG. 1A;

FIG. 4A is a perspective view of an alternate embodiment of a bone engaging assembly according to the present disclosure;

FIG. 4B is an end view of the bone engaging assembly of FIG. 4A;

FIG. 4C is a side cross-sectional view of the bone engaging assembly of FIG. 4B taken along section line 4C-4C;

FIG. 5 is a perspective view of a first bone engaging member of the bone engaging assembly of FIG. 4A;

FIG. 5A is a perspective view of an alternate embodiment of the first bone engaging member of FIG. 5;

FIG. 6 is a perspective view of a second bone engaging member of the bone engaging assembly of FIG. 4A;

FIG. 7A is an end view of the bone engaging assembly of FIG. 1A attached to adjacent vertebrae;

FIG. 7B is a partial, side, cross-sectional view of the bone engaging assembly and vertebrae of FIG. 7A illustrating an intervertebral cage disposed between the adjacent vertebrae;

FIG. 8 is a perspective view of a coupler according to a further embodiment of the present disclosure;

FIG. 9 is a perspective anterior view of one embodiment of a bone engaging assembly attached to adjacent vertebrae, the bone engaging assembly including the coupler of FIG. 8;

FIG. 10 is a perspective view of the bone engaging assembly of FIG. 4A shown attached to adjacent vertebrae;

FIG. 11 is a side view of FIG. 10;

FIG. 11A is a side, cross sectional view of a further embodiment of a bone engaging assembly;

FIG. 11B is a perspective view of the bone engaging assembly of FIG. 11A;

FIG. 12A is a side view of one embodiment of a bone engaging assembly in accordance with the present disclosure;

FIG. 12B is a end view of the bone engaging assembly of FIG. 12A;

FIG. 12C is a cross-sectional perspective view with the bone engaging assembly of FIGS. 12A and 12B shown attached to adjacent vertebrae;

FIG. 12D is a perspective view of FIG. 12C;

FIG. 12E is a rear, perspective view of FIG. 12C;

FIG. 13A is a perspective view of another embodiment of a bone engaging assembly shown attached to adjacent vertebrae;

FIG. 13B is a top, cross-sectional, perspective view of FIG. 13A;

FIG. 13C is a side, perspective view of a first bone engaging member of the bone engaging assembly of FIGS. 13A and 13B, without the rod receiving member;

FIG. 13D is a top view of the first bone engaging member of FIG. 13C;

FIG. 14A is a side, perspective view of an alternate embodiment of a bone engaging assembly shown attached to adjacent vertebrae in accordance with the present disclosure;

FIG. 14B is a side, partial cross-sectional view of FIG. 14A;

FIG. 14C is a perspective view the bone engaging assembly of FIGS. 14A and 14B;

FIG. 14D is an end view of one embodiment of a collar of the bone engaging assembly of FIGS. 14A-14C.

### DETAILED DESCRIPTION OF THE EMBODIMENTS

Particular embodiments of the present disclosure will be described herein with reference to the accompanying drawings. As shown in the drawings and as described throughout the following description, and as is traditional when referring to relative positioning on an object, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is farther from the clinician during proper use. In addition, the term “cephalad” or “cranial” is used in this application to indicate a direction toward a patient's head, whereas the term “caudad” indicates a direction toward the patient's feet. Further still, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front. In the following description, well-known functions or constructions are not described in detail to avoid obscuring the present disclosure in unnecessary detail.

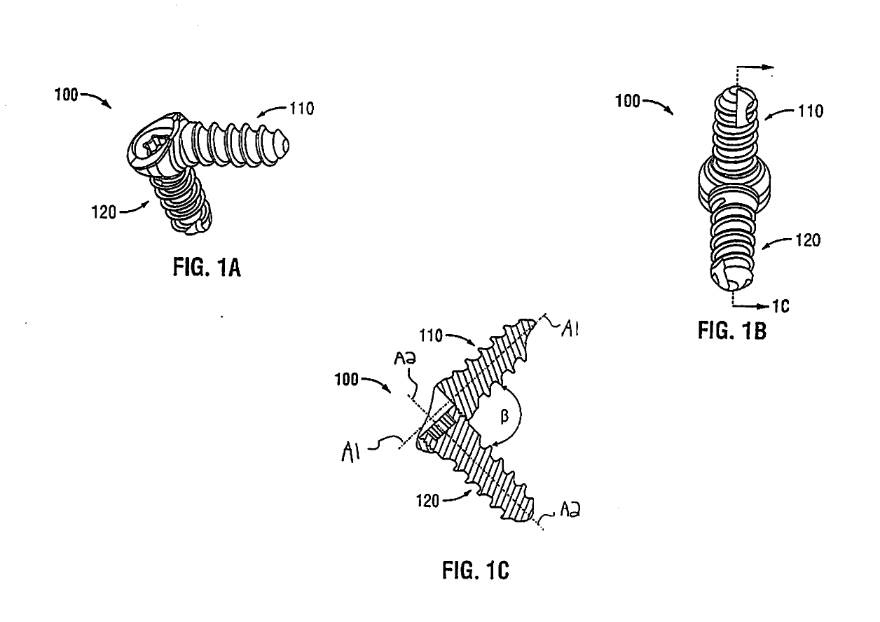

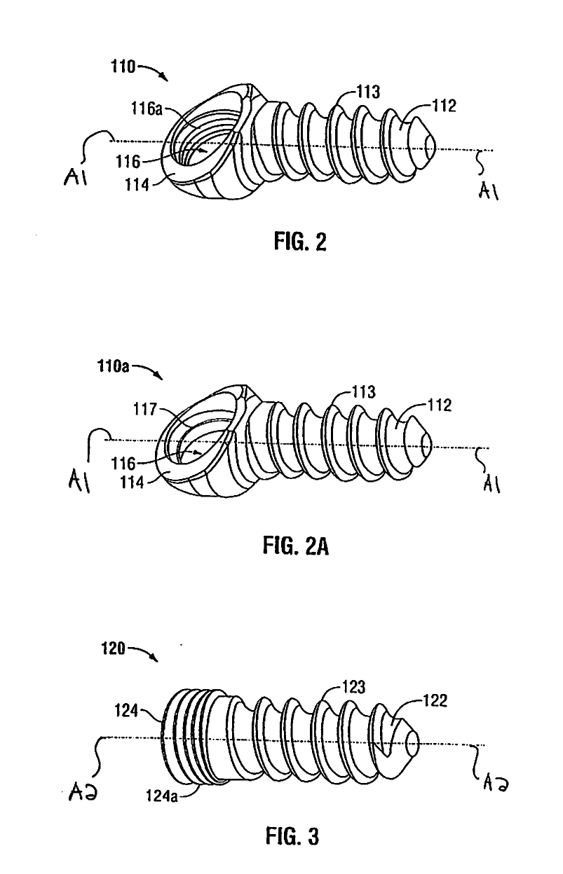

Referring initially to FIGS. 1-3, an embodiment of the presently disclosed bone engaging assembly or bone screw assembly is shown and generally identified as 100. The bone engaging assembly 100 includes a first bone engaging member or a first bone screw 110 (FIGS. 2 and 2A) and a second bone engaging member or second bone screw 120 (FIG. 3). The first bone screw 110 includes a shaft 112 and a head 114 attached thereto. The shaft 112 includes helical threads 113 peripherally disposed around the outer surface thereof. The threads 113 may adapted for threadably mating with cortical bone or with cancellous bone. The shaft 112 of the first bone screw 110 defines a first longitudinal axis A1. A bore 116 extends through the head 114 of the first bone screw 110. The bore 116 is configured and dimensioned to accommodate at least a portion of the second bone screw 120 such that the first and second bone screws 110, 120 are coupled with each other as will be explained in further detail hereinbelow. The bore 116 of first bone screw 110 may include bore threads 116a (FIG. 2). Alternatively, first bone screw 110a may include a lip 117 (FIG. 2A). The bone screw 110 may be formed from any suitable biocompatible material such as titanium, titanium alloys, PEEK, or stainless steel. It is contemplated that all or a portion of the bone screw 110 may be formed from a resorbable material, as is known in the art. Further still, the bone screw 110 may be formed of several materials including metallic and polymeric materials. It is contemplated that the head 114 of the first bone screw 110 which includes bore 116 may be radially offset from or angulated relative to the shaft 112.

The second bone screw 120 includes a shaft 122 and a head 124 attached thereto. The shaft 122 includes threads 123 peripherally disposed around the outer surface thereof. The threads 123, like threads 113 may adapted for threadably mating with cortical bone or with cancellous bone. It is contemplated that the shafts of the presently disclosed bone screws may be expandable or curved. The second bone screw 120 defines a second longitudinal axis A2. The first and the second longitudinal axes A1, A2 may be disposed at an angle β relative to each other. The angle β may be between about 0° to about 180°. In one embodiment, the angle β is about 45°. In another embodiment, the angle β is about 90°. In yet another embodiment, the angle β may be between about 30° and about 150°, while in a further embodiment, the angle β may be between about 60° and about 120°. The head 124 of the second bone screw 120 includes head threads 124a for engaging the bore threads 116a of the head 114 of the first bone screw 110 and attaching the first bone screw 110 to the second bone screw 120 as will be discussed in detail hereinbelow. The pitch of the threads 123 on the shaft 122 of the second bone screw 120 when compared to the pitch of the head thread 124a on the head 124 of the second bone screw 120 can vary such as to allow for compression across the disk space. The head 124 may include a driver interface defined therein or projecting from an outer surface thereof for engaging with a driving tool, e.g. a screwdriver. The driver interface may be any suitable shape including circular, semi-circular, hexagonal, polygonal, etc.

The head 114 of the first bone screw 110 is configured for receiving the second bone screw 120 such that the first and second bone screws 110, 120 are affixed to each other. That is, the head 114 couples the first bone screw 110 with the second bone screw 120. The lip 117 of the head 114 of the first bone screw 110a preferably is formed from commercially pure titanium. The threads 124a on the exterior of the head 124 of the second bone screw 120 preferably are formed from a titanium alloy such as Ti-6Al-4V, which is harder than the commercially pure titanium of the lip 117. As such, since the commercially pure titanium of the lip 117 is softer than the Ti-6Al-4V alloy of the threads 124a of the screw head 124, the threads 124a engage the lip 117 as the head 124 of the second screw 120 is inserted through the bore 116, thereby inhibiting the second screw 120 from separating from the first screw 110a. It is further contemplated that alternate structures may be used to affix the first and second bone screws 110, 120. These alternate structures include clips, clamps, snaps, adhesives, etc. Alternatively, the threads 116a, 124a may be complementary for forming a secure attachment for the first and second bone screws 110, 120. Each head 114, 124 may be symmetrically or asymmetrically disposed relative to one or more of the shafts 112, 122 of the respective first and second bone screws 110, 120.

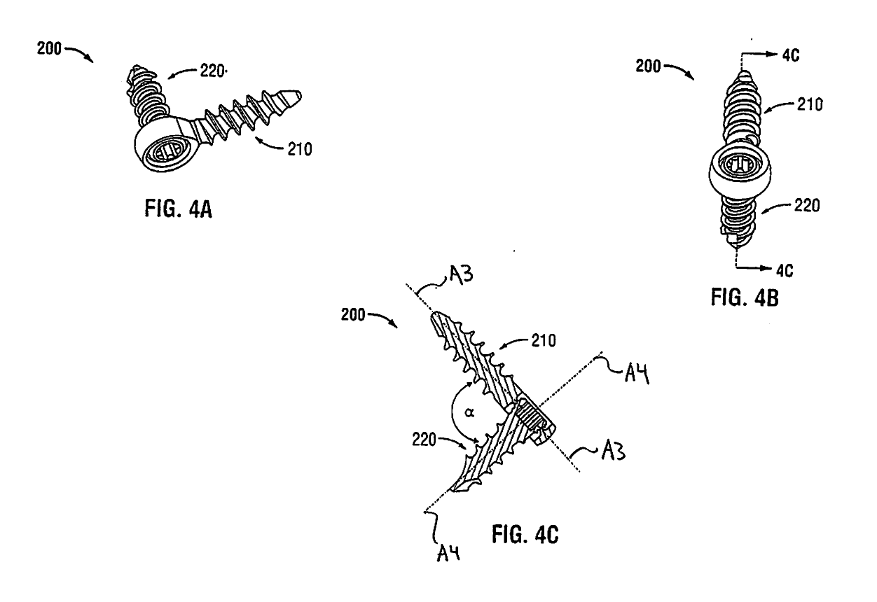

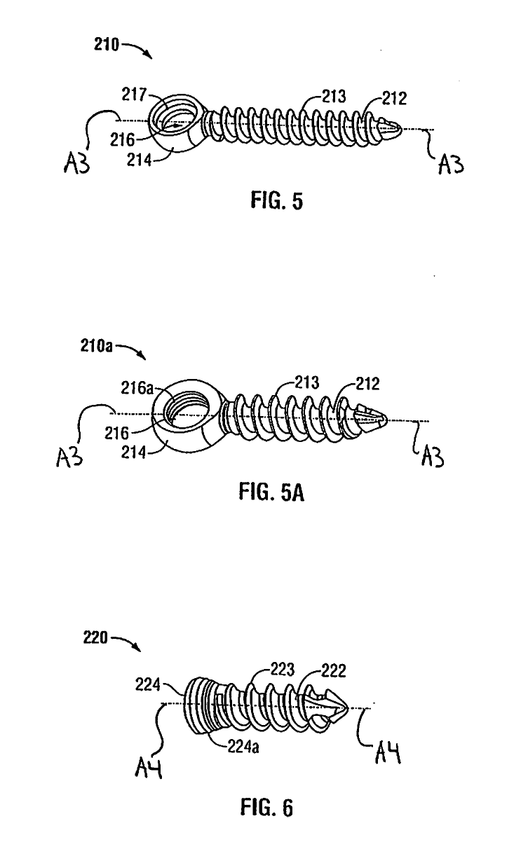

An alternate embodiment of a bone engaging assembly or bone screw assembly is illustrated in FIGS. 4-6 and generally identified as bone screw assembly 200. Bone screw assembly 200 is substantially similar to bone engaging assembly 100 and is described herein only to the extent necessary to describe the differences in construction and operation. Bone screw assembly 200 includes bone screws 210, 220. Bone screw 210 includes a shaft 212 with helical threads 213 formed on an outer surface thereof. A head 214 is attached to one end of the shaft 212. A bore 216 is extends through the head 214 of bone screw 210 and includes a lip 217 (FIG. 5) formed on an inner surface of the bore 216. Alternatively, the bore 216 of bone screw 210a may include threads 216a (FIG. 5A) formed on an inner surface thereof. The shaft 212 of the bone screw 210 defines a longitudinal axis A3, while the shaft 222 of bone screw 220 defines a longitudinal axis A4. The longitudinal axes A3, A4 define an angle α therebetween. As shown, the angle α is about 90°, although other angular relationships are within the scope of the present disclosure. Bone screw 220 includes a shaft 222 with helical threads 223 formed on an outer surface thereof. A head 224 is attached to one end of the shaft 222. The head 224 includes threads 224a formed on an outer surface thereof. The pitch of the threads 223 on the shaft 222 of the second bone screw 220 when compared to the pitch of the head thread 224a on the head 224 of the second bone screw 220 can vary such as to allow for compression across the disk space. The head 224 may include a driver interface defined therein or projecting from an outer surface thereof for engaging with a driving tool, e.g. a screwdriver. The driver interface may be any suitable shape including circular, semi-circular, hexagonal, polygonal, etc.

The lip 217 of the head 214 of the first bone screw 210 preferably is formed from commercially pure titanium. The threads 224a on the exterior of the head 224 of the second bone screw 220 preferably are formed from a titanium alloy such as Ti-6Al-4V, which is harder than the commercially pure titanium of the lip 217. As such, since the commercially pure titanium of the lip 217 is softer than the Ti-6Al-4V alloy of the threads 224a of the screw head 224, the threads 224a engage the lip 217 as the head 224 of the second screw 220 is inserted through the bore 216, thereby inhibiting the second screw 220 from separating from the first screw 210 (and thus cold welded together). It is further contemplated that alternate structures may be used to affix the first and second bone screws 210, 220. These alternate structures include clips, clamps, snaps, adhesives, etc. Alternatively, the threads 216a, 224a may be complementary for forming a secure attachment for the first and second bone screws 210a, 120. Each head 214, 224 may be symmetrically or asymmetrically disposed relative to one or more of the shafts 212, 222 of the respective first and second bone screws 210, 220. Advantageously, the lip-thread interlocking arrangement of the embodiment of FIGS. 4-6 permits the second screw to engage the bore a variety of angles, such that the axes A1 and A2 may be disposed at an angle relative to each other, providing greater flexibility to the surgeon during insertion of the screws into bone. An example of this locking arrangement is disclosed in U.S. Pat. No. 6,322,562 to Wolter, the entire contents of which are hereby incorporated by reference.

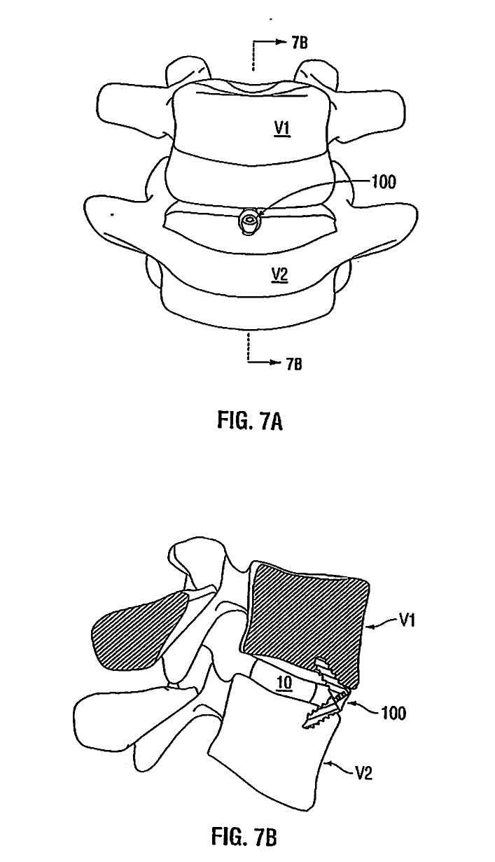

Referring additionally to FIGS. 7A and 7B, the bone screw assembly 100 is coupled to adjacent vertebrae V1, V2. Although only bone screw assembly 100 is illustrated and discussed with respect to vertebrae V1, V2, it is within the scope of the present disclosure that any of the presently disclosed bone engaging assemblies or bone screw assemblies may be used with vertebrae V1, V2 in lieu of bone screw assembly 100, or that multiple bone screw assemblies may be used between adjacent vertebrae V1, V2. The first bone screw 110 is inserted into a previously drilled hole extending into one of the vertebrae V1, V2. Alternatively, the bone screw 110 may include self-starting threads such that little or no pre-drilling is required. The shaft 122 of the second bone screw 120 is directed through the bore 116 of the first bone screw 110 with the threads 123 of the second bone screw 120 engaging a previously drilled hole extending into one of the vertebrae V1, V2. Alternatively, the second bone screw 120 may include self-starting threads such that little or no pre-drilling is required. After insertion of the second bone screw 120, the heads 114, 124 of the first and second bone screws 110, 120 are then interlocked, as discussed hereinabove, such that the first and second bone screws 110, 120 are disposed at an angle β relative to each other. The bone screws 110, 120 are selected such that the respective shafts 112, 122 have sufficient length such that the bone screws 110, 120 are securely affixed to their respective vertebrae V1, V2 (i.e. sufficient purchase into bone tissue). Further still, since the bone screws 110, 120 are inserted at a preselected angle and the head 124 of bone screw 120 is securely affixed to the head 114 of bone screw 110, both bone screws 110, 120 resist separation from vertebrae V1, V2. As such, the first and second bone screws 110, 120 affix the first and second vertebrae V1, V2 in a relative relationship with each other. It is contemplated that an intervertebral implant 10 may be disposed between the vertebrae V1, V2 prior to attaching the bone screw assembly 100, 200 to the vertebrae V1, V2. It is contemplated that the intervertebral implant 10 may be retained in position by the bone screw assembly 100, 200 or that it may be threadably engaged with the bone screw assembly 100, 200. It is contemplated that the intervertebral implant 10 may include one or more openings for receiving the shafts of the screws of the bone screw assembly. The openings in the intervertebral implant may be complementary to the thread of the bone screws of the selected bone screw assembly. Alternatively, the interbody implant may be contoured to nest with the screw assembly.

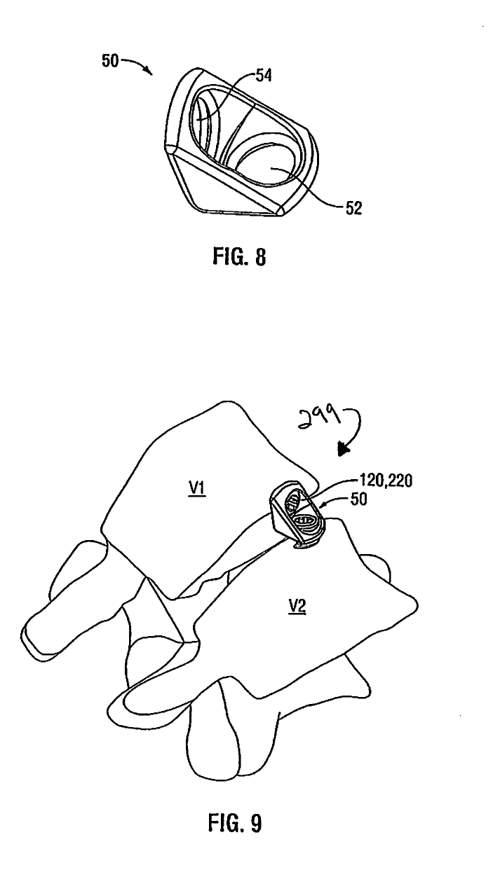

Referring now to FIGS. 8 and 9, in an alternate embodiment of a bone engaging assembly 299, which is substantially similar to bone engaging assembly 100, is described herein only to the extent necessary to describe the differences in construction and operation. Bone engaging assembly 299 includes a coupler 50 (FIG. 8) that includes first and second bores 52, 54 defined therethrough. Each bore 52, 54 is adapted to receive a bone screw 110, 120, 210, 220 for mounting to vertebrae V1, V2 and includes respective lips 52a, 54a. First and second bores 52, 54 are shown disposed in substantially orthogonal relationship. However, first and second bores 52, 54 may be disposed at any suitable angle relative to one another for receiving bone screws therethrough. Similar to the fastening arrangement discussed hereinabove with respect to bone screw assembly 100, 200, at least one of the lips 52a, 54a preferably is formed from commercially pure titanium. When the second bone screw 120, 220 is inserted through the bore 52, 54 and engages the lip 52a, 54a that is formed from commercially pure titanium, the threads 114a, 124a that are formed from a harder titanium alloy such as Ti-6Al-4V engage the threads 114a, 124a and affix the bone screw 120, 220 to the bore 52, 54 of the coupler 50. In this arrangement, the bone screw 120, 200 is resistant to backing out of the bore 52, 54. Bone screw 110, 210 is inserted through the other bore 52, 54 and into the bone of the vertebral body. When the coupler 50 is assembled with bone screws 120, 220, the assemblage fixates the adjacent vertebrae V1, V2 with respect to each other. It is also envisioned that when bone screws 120, 220 are installed in the coupler 50, one bone screw 120, 220 may be partially threaded into the bore 52, 54 such that the head 124, 224 covers the head 124, 224 of the remaining bone screw 120, 220, thereby limiting the distance the remaining bone screw 120, 220 can travel in the event it starts to back out of the coupler 50. It is contemplated that the connector may be configured to nest with an interbody implant or with a bone plate mounted to the vertebral bodies above and below the intervertebral space. It is also contemplated that the presently disclosed coupler may have a hinge located between the bores or that the coupler is flexible such that the angular relationship between the bores is adjustable. Furthermore, coupler 50 may used to facilitate the positioning of bone screw 120 into a facet joint and into a pedicle of a caudad vertebra or a facet joint, pedicle, and vertebral body of a caudad vertebra. The coupler 50 may also be used to facilitate the positioning of bone screw 220 into the pars interarticularis or into the par interarticularis and the pedicle of a cranial vertebra.

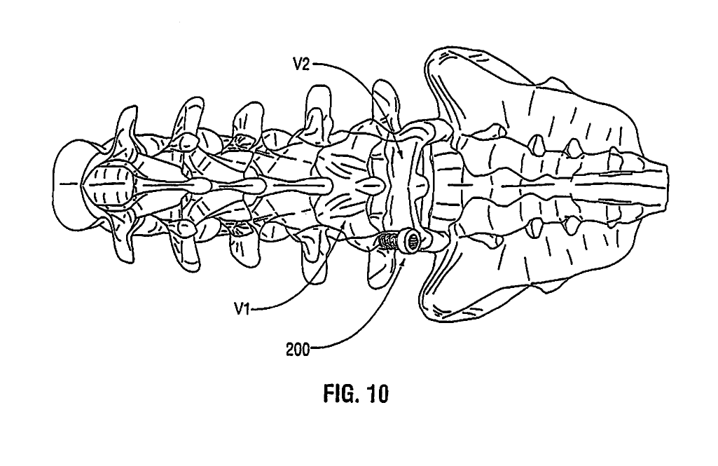

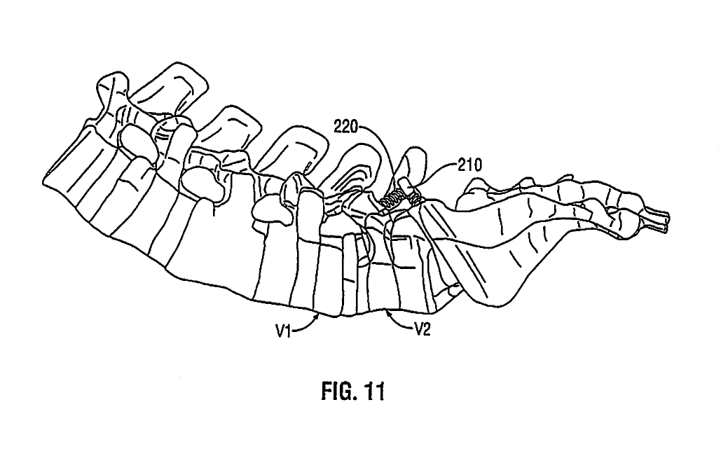

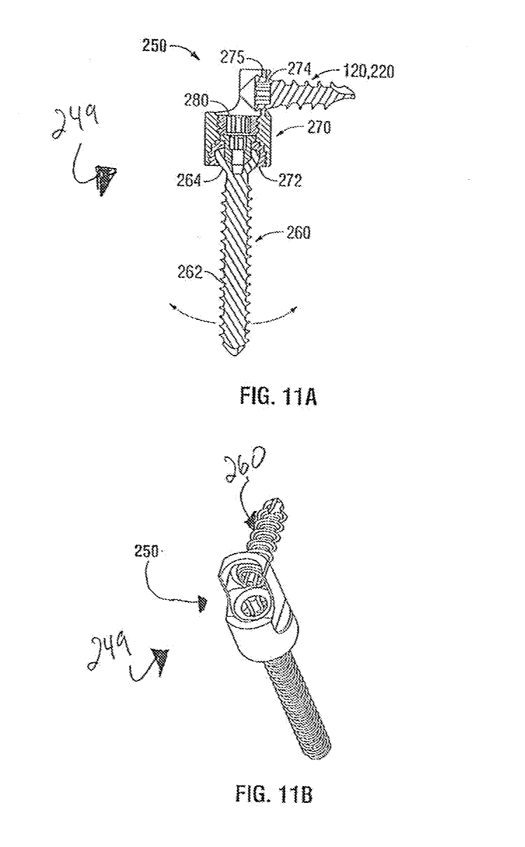

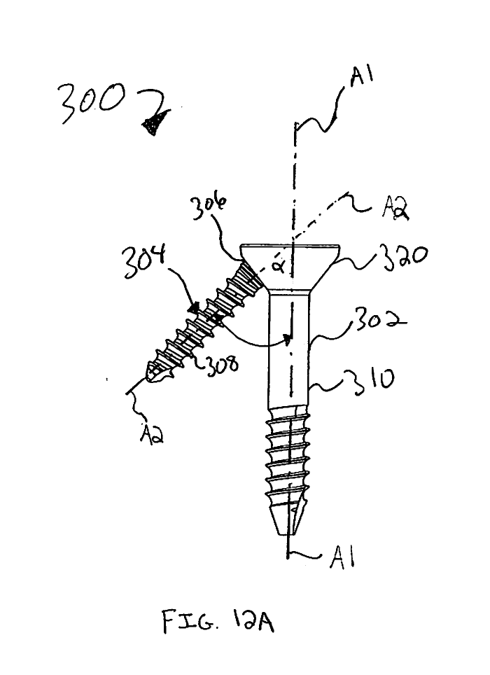

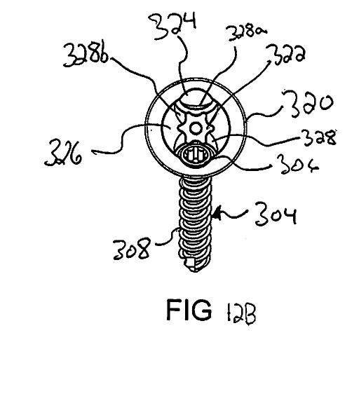

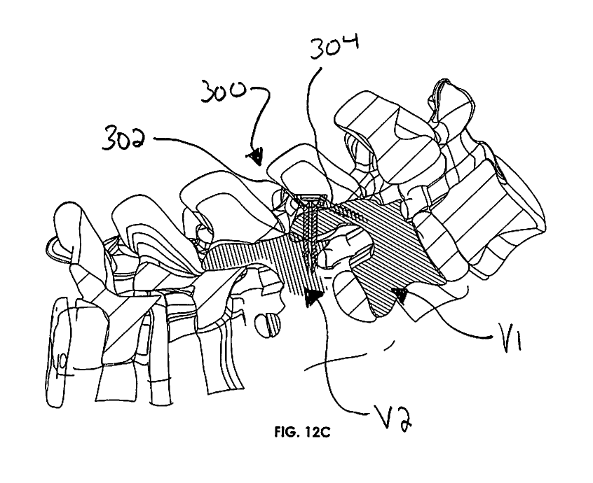

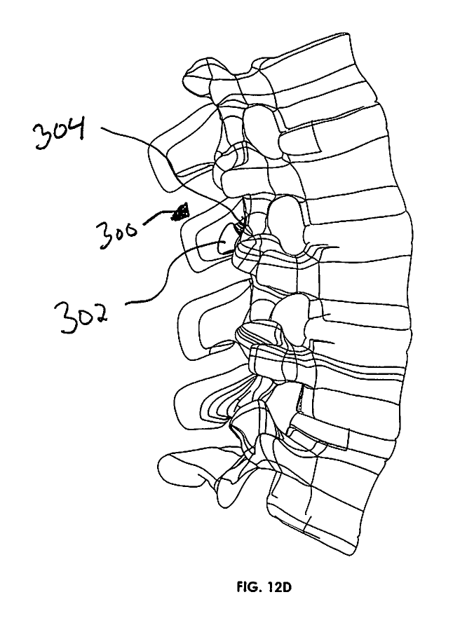

Any of the presently disclosed bone screw assemblies are capable of being used for transfacet fixation. Referring additionally to FIGS. 10 and 11, bone screw 210 is inserted into the pars interarticularis PA. Bone screw 220 is inserted through head 214 of bone screw 210 and inserted into the facet joint. Alternatively, bone screw 210 may be inserted into the facet joint and bone screw 220 is received through head 214 of bone screw 210 and secured to the pars interarticularis PA. It is contemplated that either bone screw 210 or 220 may be inserted towards the midline into the spinolaminar junction or spinous process or lamina. These sites would provide additional bony purchase on the vertebral body. It is envisioned that the bone screw 210 and any of the presently disclosed first or second bone engaging members may be inserted through a pre-drilled hole or may have self-starting threads formed thereon as discussed hereinabove. Since the pars interarticularis PA is formed from cortical bone, it provides a much more secure purchase for the bone screw 210. Once bone screw 210 is securely anchored in the pars interarticularis PA, the other bone screw 220 is inserted through the head 214 of bone screw 210 and affixed through the facet joint as discussed hereinabove. The location of the installation of the bone screw assembly 200 into the pars interarticularis PA provides a secure anchor point. As such, this arrangement provides a more stable fixation arrangement for adjacent vertebrae V1, V2 with respect to each other than provided by using a transfacet screw alone. Further still, this arrangement allows the practitioner to build other constructs. It is also contemplated that the first screw may be mounted through the inferior facet of a cranial vertebra, through the superior facet joint and into the pedicle of the adjacent caudal vertebra, with the second screw mounted into the pars interarticularis of the cranial vertebra.

Alternatively, with reference to FIG. 11A, another embodiment of a bone engaging assembly 249 includes a first bone engaging member or a bone anchor 250 and a second bone engaging member or a bone screw 260. Bone anchor 250 includes a polyaxial bone screw 260 and a coupling member 270. The bone screw 260 includes a head 264 and a shaft 262. The shaft 262 includes threads 263 peripherally disposed on an outer surface thereof. The head 264 has an arcuate portion that is proximal to the shaft 262. The bone screw 260 is rotatable relative to the coupling member 270 and is also repositionable such that a plurality of angular relationships may be defined between the bone screw 260 and the coupling member 270. The coupling member 270 includes a plurality of openings 272, 274 extending therethrough. Opening 272 is configured and dimensioned for receiving the head 264 of the bone screw 260 therethrough such that the head 264 is pivotably disposed in the opening 272. The coupling member 270 further includes threads 273 formed on an inner surface thereof for threadably engaging a set screw 280. As set screw 280 is threaded towards the head 264 of the bone screw 260, it frictionally engages the head 264 and secures the bone screw 260 in a set orientation relative to the coupling member 270. The other opening 274 is disposed orthogonally to opening 272. Opening 274 includes a lip 275 formed on an inner surface thereof. Bone screw 120 or bone screw 220 is insertable through the opening 274. The lip 275 preferably is formed from commercially pure titanium. The threads 124a or 224a on the exterior of the head 124 or 224 of the bone screw 120 or 220 are formed from a titanium alloy such as Ti-6Al-4V, which is harder than the commercially pure titanium of the lip 275. As such, since the commercially pure titanium of the lip 275 is softer than the Ti-6Al-4V alloy of the threads 124a, 224a, the threads 124a, 224a engage the lip 275 as the head 124, 224 of the bone screw 120, 220 is inserted through the opening 274, thereby inhibiting the bone screw 120, 220 from separating from the bone anchor 250. As constructed, bone anchor 250 is insertable into a selected bone structure, such as a vertebral body, and the coupling member 270 is pivotable relative to the anchor location of the bone screw 260 allowing the coupling member 270 to be pivoted and/or rotated such that opening 274 is in a desired orientation to the target bone structure. Bone screw 120 or 220 is inserted through the opening 274 and affixed to the target bone structure. The head 124 or 224 is secured to the lip 275 of the opening 274 as discussed above. As such, the target bone structure is fixedly coupled to the selected bone structure. It is contemplated that the bone structures may include pars interarticularis, spinolaminar junction, spinous process, or lamina.

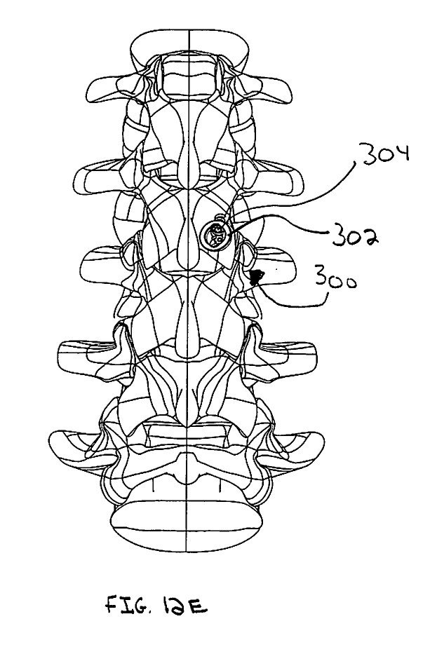

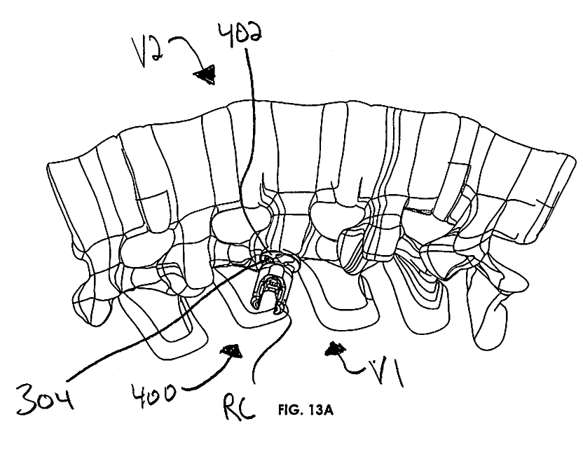

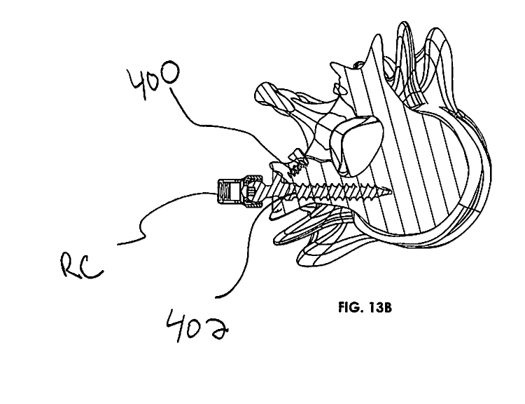

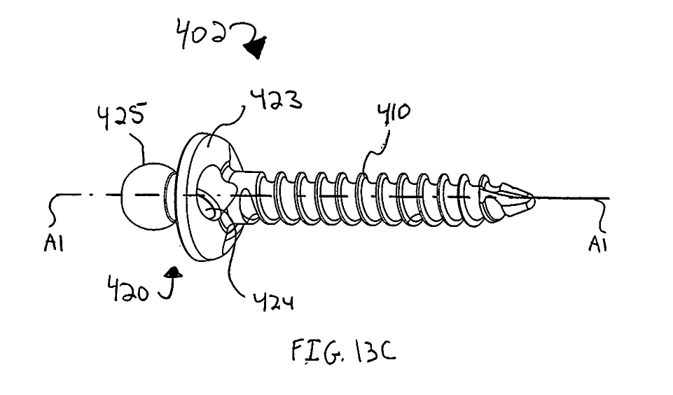

As illustrated in FIGS. 12A-12B, one embodiment of a bone engaging assembly or a bone screw assembly is generally referred to 300. Bone engaging assembly 300 is substantially similar to bone engaging assembly 100 and is described herein only to the extent necessary to describe the differences in construction and operation. Bone engaging assembly 300 includes a first bone engaging member 302, (e.g., a first bone screw) and a second bone engaging member 304 (e.g., a second bone screw). The second bone engaging member 304 defines a second axis A2 and a length. The second bone engaging member 304 includes a second head 306 and a second shaft 308. The second shaft 308 is at least partially threaded. The first bone engaging member 302 defines a first axis A1 and a length. The lengths of the first and second bone engaging members 302, 304 may be different. The first bone engaging member 302 has a first shaft 310 and a first head 320. The first shaft 310 is at least partially threaded.

As best depicted in FIG. 12B, the first head 320 defines a driving interface 322 and one or more apertures 324. The apertures 324, which are described in greater detail below, extend through the first head 320. The driving interface 322 extends partially into the first head 320 and is configured to engage a driving instrument, e.g., a screwdriver (not shown). With reference again to FIG. 12A, the first axis AI of the first bone engaging member 302 and the second axis A2 of the second bone engaging member 304 define an angle α therebetween when the second bone engaging member 304 is positioned within one of the apertures 324 of the first head 320. The first bone engaging member 302 may be oriented at an acute angle relative to the second bone engaging member 304.

As illustrated in FIG. 12A, the second head 306 may be in contact with the first head 320 when the second bone engaging member 304 is positioned within one of the apertures 324 of the first head 320. The apertures 324 are positioned at an angle relative to the first axis A1 of the first bone engaging member 302 and in parallel with the second axis A2 of the second bone engaging member 304 when the second bone engaging member 304 is positioned within one of the apertures 324. Referring again to FIG. 12B, the first head 320 defines a first surface 326 and one or more second surfaces 328. The second surfaces 328 project substantially parabolically from the first surface 326. The second surfaces 328 may project proximally and/or distally from the first head 320. In this respect, one or more pairs of second surfaces 328 may extend in opposed relationship on the first head 320 such that the aperture 324 defined between the opposed pair of second surfaces 328 forms a substantially paraboloid shape (e.g., elliptical or hyperbolic). In this regard, the one or more opposed pairs of second surfaces 328 may be substantially mirrored across coronal, sagittal and/or transverse planes (not shown) defined relative to the first head 320. The second surfaces 328 define an internal section 328a and an external section 328b. Boundaries of the internal section 328a define at least a portion of one of the respective apertures 324.

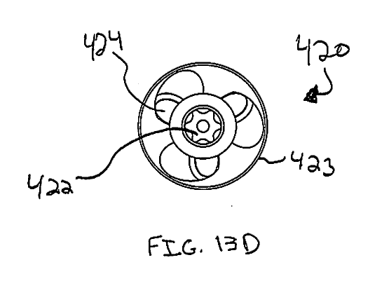

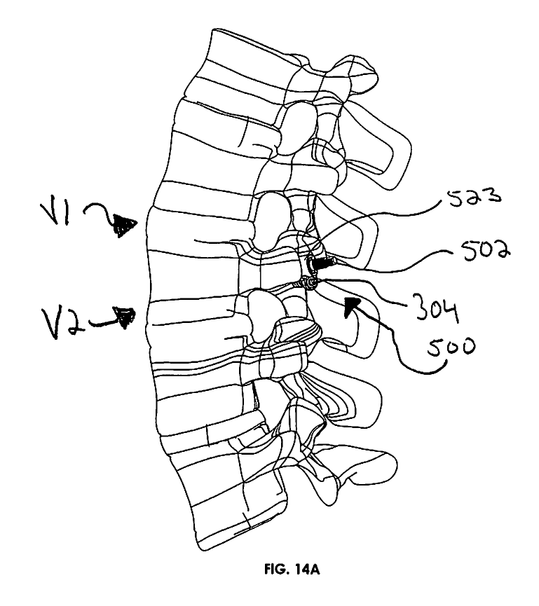

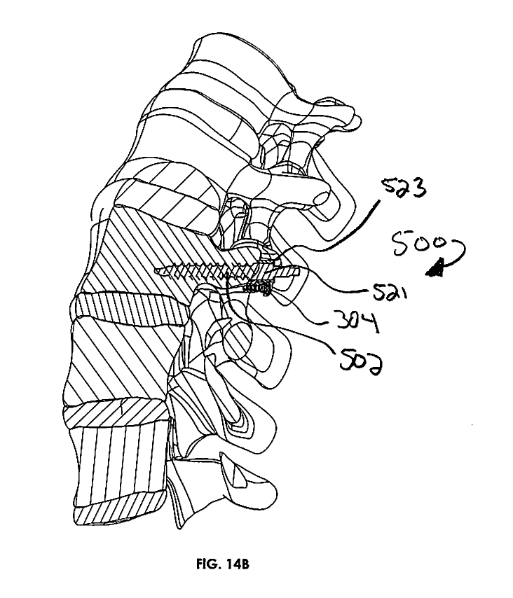

FIGS. 13A-13B show an alternate embodiment of a bone engaging assembly which is generally referred to as 400. Bone engaging assembly 400 is substantially similar to bone engaging assembly 300 and is described herein only to the extent necessary to describe the differences in construction and operation. Bone engaging assembly 400 includes a first bone engaging member 402 (e.g., a bone anchor) and a second bone engaging member 304 (e.g., a bone screw). As best depicted in FIG. 13C, the first bone engaging member 402 defines a first axis A1. The first bone engaging member 402 has a first shaft 410 and a first head 420. The first head 420 includes a substantially spherical portion 425 that is configured to engage a rod coupling member “RC” (see FIG. 13A) such as a rod receiving tulip. With reference to FIG. 13D, first head 420 defines a driving interface 422 for engagement with a driving instrument, e.g., a screwdriver. First head 420 also includes a collar 423 that is integrally formed with the first head 420. As shown in FIG. 13C, the collar 423 is longitudinally spaced from the driver interface 422 along the first axis A1. As illustrated in FIG. 13D one or more apertures 424 (substantially similar to apertures 328) are defined within the collar 423 and are positioned radially about the collar 423.

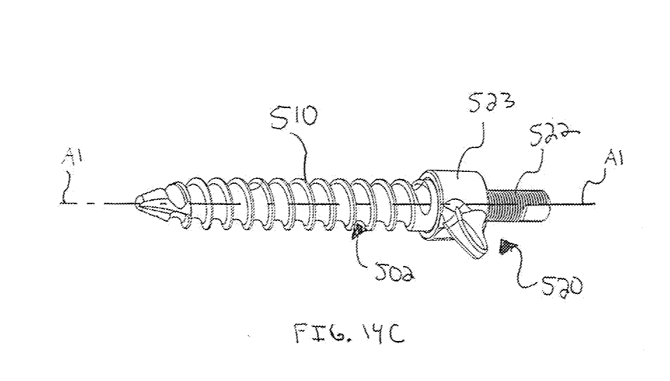

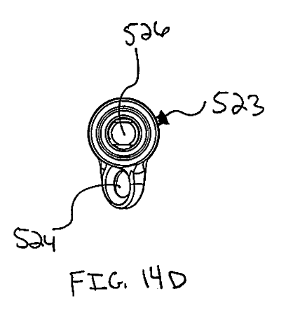

FIGS. 14A-14B show an alternate embodiment of the bone engaging assembly which is generally referred to as 500. Bone engaging assembly 500 is substantially similar to bone engaging assembly 300 and is described herein only to the extent necessary to describe the differences in construction and operation. As depicted in FIGS. 14B and 14C, bone engaging assembly 500 includes a first bone engaging member 502 and a second bone engaging member 304. The first bone engaging member 502 is a bone anchor and defines a first axis A1. The first bone engaging member 502 has a first shall 510 and a first head 520. The first head 520 includes a threaded post 522 extending proximally from a mounting portion 521 (FIG. 14B). With brief reference to FIG. 14B, the mounting portion 521 defines a taper (e.g., a Morse taper). The threaded post 522 is configured to engage a rod coupling member “RC” (see, e.g., FIG. 13A) such as rod receiving tulip. The first head 520 includes a collar 523 that is selectively attachable to the mounting portion 521 of the first head 520 or permanently attached to the mounting portion 521. Referring now to FIG. 14D, the collar 523 defines one or more apertures 524 therethrough and a passage 526 therethrough. The passage 526 facilitates the securement of the collar 523 to the first head 520 whereby the post 522 extends proximally beyond the passage 526. As shown in FIGS. 14C and 14D, aperture 524 may be positioned at an angle relative to the passage 526.

In embodiments, at least a portion of one of the presently disclosed first and second bone engaging members may be made of commercially pure titanium and at least a portion of the other of the first and second bone engaging members may be made of titanium alloy.

The devices disclosed herein may be used in a number of novel surgical methods.

First, as shown in FIGS. 7A-7B, the screw constructs disclosed herein may be used to secure an interbody implant against dislocation. While FIGS. 7A-7B illustrate a single pair of interlocking screws disposed across the disc space, it is contemplated that multiple screw pairs may be inserted at spaced locations across the anterior of the spine to provide greater security and support of the adjacent vertebrae V1, V2 during the fusion process. Similarly, multiple brackets of the type shown in FIGS. 8-9 may be used.

Second, the screw constructs may be used in novel procedures to fix one vertebral level relative to another. In this regard:
1. A first screw may be inserted through the inferior facet at the cranial vertebral level into the superior facet of the adjacent caudal vertebral level, with a second screw mounted through the head of the first screw into additional structure of the cranial level, such as the pars interarticularis, the spinal-laminar junction or the lamina.
2. As a variation, the first screw may be inserted through the inferior facet at the cranial level into the superior facet of the adjacent caudal spinal level and into the pedicle of the caudal level, with a second screw mounted through the head of the first screw into additional structure of the cranial level, such as the pars interarticularis, the spinal-laminar junction or the lamina.
3. As yet a further variation, the first screw may be inserted through the inferior facet at the cranial level into the superior facet of the adjacent caudal spinal level, into the pedicle of the caudal level, and into the vertebral body at the caudal level, with a second screw mounted through the head of the first screw into additional structure of the cranial level, such as the pars interarticularis, the spinal-laminar junction or the lamina

Alternatively, the first screw could be placed into the cranial structure (pars interarticularis, spinal-laminar junction, lamina, etc.) with the second screw inserted through the aperture in the head of the first screw into the facet joint, facet joint and pedicle or facet joint, pedicle and vertebral body of the caudal level. Any of these variations may be used for spinal fixation between two adjacent spinal levels. The method may be repeated at multiple spinal levels. Such a procedure may be used by itself for spinal fixation or may be used in conjunction with additional fusion techniques such as interbody fusion.

Third, the screw constructs may be used at a single spinal level to reinforce a screw-rod construct. In this regard, the first screw may be inserted into a pedicle with the second screw inserted through the head of the first screw into additional bone structure for supplemental fixation. The second screw preferably is inserted into the pars interarticularis of the same spinal level as the pedicle into which the first screw has been inserted. Alternatively, the second screw may be inserted into the spinal-laminar junction or the lamina. The first screw preferably is provided with a rod coupling member. The first screw may be a polyaxial type screw or a posted screw to which a rod coupler may be mounted. In a further variation, the first screw may be mounted to the sacrum, with the second screw mounted through the first screw into the sacral ala. The foregoing constructs may find particular application in providing supplemental fixation where the first screw is inserted into poor quality bone, such as osteoporotic bone. In such case, the second screw mounted into the cortical bone of the pars interarticularis, spinal-laminar junction or lamina provides significantly enhanced screw fixation and resistance to screw back out or loosening of the first screw from bone. Such constructs may also find application to reinforcement at points of high stress concentration in a rod-screw construct, regardless whether poor bone is involved, such as at the end of a long construct. Thus, the first screw could be inserted into the pedicle of a lumbar vertebra at the end of a construct, with the second screw inserted through and secured to the first screw and mounted into another bony structure, such as the pars interarticularis, spinal-laminar junction or lamina. Alternatively, the end of construct screw could be inserted into the sacrum, with the second screw mounted through the head of the first screw into the sacral ala. In these instances, the second screw provides supplemental fixation against the high stress concentrations experienced at the end of a long construct, and resistance to first screw back out or loosening.

In use, standard techniques of drilling, tapping, and probing pedicle screw holes can be used for insertion of one of the presently disclosed first bone engaging members. As illustrated in FIGS. 12C-12E, one of the presently disclosed first bone engaging members 302 may be inserted through the medial, superior side of the facet joint of a cranial vertebra V1 and advanced laterally through the facet joint and into the pedicle of the caudal vertebra V2. The first bone engaging member 302 may also be further advanced into the vertebral body of the caudad vertebra V2. A “pineapple reamer”, as is known in the art, may be used to provide a working area to accommodate the collar and allow for the seating of the collar at the appropriate depth. As shown in FIG. 12C, second screw 304 is inserted through an aperture in the head of the first screw 302 and inserted into additional boney structure at the cranial level V1, such as the pars interarticularis.

The pars interarticularis of a cranial vertebra V1 may then be drilled into for facilitating the anchoring of one of the presently disclosed second bone engaging members 304 to provide additional support, which may be especially necessary where the first bone engaging member (e.g., a pedicle screw) is positioned into osteoporotic bone. Incremental drilling may be necessary to avoid drilling beyond the limits of the pars interarticularis and entering into the vertebral canal. The pars interarticularis can be up to 15 mm thick. Thus, in order to achieve at least 7 mm of cortical thread purchase into the pars interarticularis of the cranial vertebra V1, the pars interarticularis of the cranial vertebra V1 may be drilled with a 5 mm long drill stop (not shown), thus creating a pilot hole. In this regard, the floor of the drilled pilot hole can be subsequently checked. The following drill stop may be 9 mm (not shown) and incremented (e.g., via 2 mm increments) until the desired depth is attained. The floor of each new depth should be checked. This stepped approach limits the risk of neurologic damage to the more medially positioned neural elements. Once the drilling has been completed, the hole is then tapped with a cortical threaded tap to the appropriate depth for the reception of one of the presently disclosed second bone engaging members 304.

The second bone engaging member 304 is advanced through the head of the first bone engaging member 302 and into the pars interarticularis of the cranial vertebra V1, thereby anchoring the second bone engaging member 304 to the pars interarticularis of the cranial vertebra V1 (e.g., for lumbar vertebrae L1 to L5). The second bone engaging member 304 may also be inserted into both the pars interarticularis and the pedicle of the cranial vertebra V1. Alternatively, or additionally, the second bone engaging member 304 may even be inserted into the lamina of the cranial vertebra V1. A pilot hole may be drilled and tapped for the second bone engaging member 304 as well. The second shank 308 of the second bone engaging member 304 may be positioned superiorly of the first shank 310 of the first bone engaging member 302.

Referring to FIGS. 13A-13B and 14A-14B, the configuration there shown may find particular application in patients with poor bone quality, including osteoporotic bone. Thus, first screw 402, 502 may be mounted to a spinal pedicle as part of a screw-rod construct for spinal fixation. In the case of poor or osteoporotic bone, mounting second screw 304 into the nearby pars interarticularis, which consists of cortical bone, may provide increased security of the screw and the screw-rod construct by providing additional purchase in cortical bone. This may provide a more secure construct than a pedicle screw alone. As part of such a construct pedicle screws or, more preferably, screw assemblies of the present disclosure mounted to pedicles with additional fixation to the pars interarticularis, may be placed at additional levels of the spine, such that the rod mounted to such screws or screw assemblies extend across multiple levels of the spine to provide spinal fixation. Additional spinal fixation may be provided in a similar manner on the opposing side of the spine, so that the spine fixation construct extends along both sides and corresponding levels of the spine.

At the S1 level, one of the presently disclosed second bone engaging members 304 are not directed medially into any pars interarticularis, but rather laterally into the sacral ala. One or two additional second bone engaging members 304 may be used to augment the first bone engaging member 302, 402, 502 such that the one or more second bone engaging members 304 are positioned in the pars interarticularis of the adjacent vertebra. The first screw may have a rod-receiving member pre-attached, or such a member may be attached after the first screw, and possibly the second screw, has been placed in bone. With regard to the bone engaging assembly 500, the collar 523 (when configured to be selectively engagable) may be selectively mounted to the mounting region 521 of the first head 520 of the first bone engaging member 502 (compressing may be necessary). Alternately, the collar 523 may be mounted prior to insertion of the first bone engaging member 502 (for either the selectively engagable or the permanently attached collar). The first head 420, 520 of one of the presently disclosed first bone engaging members 402, 502 can be adjusted so that the position of one or more apertures 424, 524 of the first head 420, 520 of the first bone engaging member 402, 502 are oriented to facilitate a desired trajectory of one of the presently disclosed second bone engaging members 304. Referring to FIGS. 14C-14D, the angle of second screw aperture may be selected to accommodate the patient's anatomy. It is contemplated that a series of collars 523 may be provided and available at the time of surgery so that the surgeon may select the collar that provides a desirable angle for insertion of the second screw into bone adjacent to the first screw. It is also contemplated that a primary screw having a rod receiving member could be mounted through the inferior facet of a cranial vertebra, into the facet and pedicle or facet, pedicle and vertebral body of the caudal vertebra, with the second screw mounted to additional boney structure at the cranial level (e.g., the pars interarticularis, spinolaminar junction, spinous process or lamina). In this manner, a primary screw so mounted may be included in a multi-level screw-rod construct.

Once the bone engaging members have been anchored, any suitable rod may be secured to rod coupling member RC (FIG. 13A) known in the art.

In any of the presently disclosed embodiments, the second bone engaging member 304 may be cold welded to the first bone engaging member upon mounting the second bone engaging member 304 to the first bone engaging member as discussed above.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A bone engaging assembly, comprising:
a first bone engaging member defining a first axis and having a first head and a first shaft, the first head defining at least one aperture therethrough and a driver interface, the driver interface defined within the first head and configured to engage a driving instrument, the first head coupled to a rod coupling member; and
a second bone engaging member defining a second axis, the first axis of the first bone engaging member and the second axis of the second bone engaging member defining an angle therebetween when the second bone engaging member is positioned within the at least one aperture of the first head.

2. The bone engaging assembly of claim 1, wherein the first bone engaging member is oriented at an acute angle relative to the second bone engaging member.

3. The bone engaging assembly of claim 1, wherein the first bone engaging member defines a first length and the second bone engaging member defines a second length, the first and second lengths being different.

4. The bone engaging assembly of claim 1, wherein the second bone engaging member defines a second head and a second shaft, the second head being in contact with the first head when the second bone engaging member is positioned within the at least one aperture.

5. The bone engaging assembly of claim 1, wherein at least a portion of one of the first and second bone engaging member is made of commercially pure titanium and at least a portion of the other of the first and second bone engaging member is made of titanium alloy.

6. The bone engaging assembly of claim 1, wherein the first head includes a collar that is integrally formed with the first head.

7. The bone engaging assembly of claim 6, wherein the at least one aperture is defined within the collar.

8. The bone engaging assembly of claim 7, further comprising a plurality of apertures defined within the collar and positioned radially about the collar.

9. The bone engaging assembly of claim 6, wherein the collar is longitudinally spaced from the driver interface along the first axis.

10. The bone engaging assembly of claim 1, further comprising a rod secured to the rod coupling member.

11. The bone engaging assembly of claim 1, wherein the at least one aperture is positioned at an angle relative to the first axis of the first bone engaging member and in parallel with the second axis of the second bone engaging member.

12. The bone engaging assembly of claim 1, wherein the first head defines a first surface and at least one second surface, the at least one second surface projecting substantially parabolically from the first surface, the at least one second surface defining an internal section and an external section, boundaries of the internal section defining at least a portion of the at least one aperture.

13. A bone engaging assembly, comprising:
a first bone engaging member defining a first axis and having a first head and a first shaft, the first head defining at least one aperture therethrough and including a post extending from the first head configured to engage a rod coupling member; and
a second bone engaging member defining a second axis, the first axis of the first bone engaging member and the second axis of the second bone engaging member defining an angle therebetween when the second bone engaging member is positioned within the at least one aperture of the first head.

14. The bone engaging assembly of claim 13, wherein the first head includes a collar that is selectively attachable to the first head, the selectively attachable collar defining the at least one aperture therethrough and a passage therethrough, the passage facilitating the securement of the collar to the first head whereby the post extends proximally of the passage.

15. The bone engaging assembly of claim 14, wherein the at least one aperture is positioned at an angle relative to the passage.

16. A method of mounting a bone engaging assembly, comprising:
providing a first bone engaging member and a second bone engaging member, the first bone engaging member including a first head and a first shank, the second bone engaging member including a second head and a second shank;
anchoring the first bone engaging member to a facet joint and a pedicle of a first vertebra;
mounting the second head of the second bone engaging member to the first head of the first bone engaging member; and
anchoring the second bone engaging member to the pars interarticularis of a second vertebra.

17. The method of claim 16, further comprising:
inserting the first bone engaging member through the inferior facet of the second vertebra, into the medial, superior side of the facet joint of the first vertebra; and
advancing the first bone engaging member laterally through the facet joint and into the pedicle of the first vertebra.

18. The method of claim 17, further comprising:
advancing the first bone engaging member into the vertebral body of the first vertebra.

19. The method of claim 17, further comprising:
advancing the second bone engaging member through the pars interarticularis into the pedicle of the second vertebra.

20. The method of claim 16, further comprising:
advancing the second bone engaging member through the first head of the first bone engaging member and into the pars interarticularis of the second vertebra.

21. The method of claim 16, further comprising:
providing the first bone engaging member with a first head including at least one aperture; and
adjusting the first head of the first bone engaging member so that the position of the at least one aperture is oriented to facilitate a desired trajectory of the second bone engaging member.

22. The method of claim 21, further comprising:
cold welding the second bone engaging member to the first bone engaging member upon mounting the second bone engaging member to the first bone engaging member.

23. The method of claim 16, further comprising:
positioning the second shank superiorly of the first shank.

24. A method of mounting a bone engaging assembly, comprising:
providing a first bone engaging member and a second bone engaging member, the first bone engaging member including a first head and a first shank, the second bone engaging member including a second head and a second shank;
anchoring the first bone engaging member to a facet joint and a pedicle of a vertebra;
mounting the second head of the second bone engaging member to the first head of the first bone engaging member; and
anchoring the second bone engaging member laterally to the sacral ala.
